(19) United States
(12) Patent Application Publication
(10) Pub. No.: US 2010/0168423 A1
(43) Pub. Date: Jul. 1, 2010

(54) METHOD FOR THE PRODUCTION OF BRIDGED DIBENZ[c,e] [1,2]-OXAPHOSPHORIN-6-OXIDES

(75) Inventors: Manfred Döring, Worth-Buchelberg (DE); Brigitte Lindner, Sargans (CH); Andreas Kaplan, Chur (CH)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: EMS-PATENT AG, Domat (CH)

(21) Appl. No.: 12/540,007

(22) Filed: Aug. 12, 2009

(30) Foreign Application Priority Data
Aug. 14, 2008 (CH) 01280/08

(51) Int. Cl. C07D 251/32

(52) U.S. Cl. 544/192

(57) ABSTRACT

The invention relates to an improved method for the synthesis of monomeric and polymeric nitrogen-bridged derivatives of dibenz[c,e][1,2]-oxaphosphorin-6-oxides. These substances can be used as flameproofing agents for polyesters, polyamides, polycarbonates, epoxy resins, inter alia polymers.

The method relates to an improved method for the synthesis of monomeric and polymeric nitrogen-bridged derivatives of dibenz[c,e][1,2]-oxaphosphorin-6-oxides. These substances can be used as flameproofing agents for polyesters, polyamides, polycarbonates, epoxy resins, inter alia polymers.

### BACKGROUND OF THE INVENTION

The production of nitrogen-containing dibenz[c,e][1,2]-oxaphosphorin-6-oxides has been achieved to date by two methods:
1. By aminomethylation of 6H-dibenz[c,e][1,2]-oxaphosphorin-6-oxides with aldehydes or ketones, preferably with formaldehyde, in the presence of primary or secondary amines, as is described for example in DE 27 30 345. The use of these compounds as flameproofing agents for various polymers is described likewise in the patent literature (U.S. Pat. No. 4,742,088; JP 2002-284850; JP 2001-323268).
2. By conversion of alkoxy-(6H)-dibenz[c,e][1,2]-oxaphosphorins with nitrogen-containing polyols and subsequent Michaelis-Arbuzov reaction (WO 2006/084488 A1). This method produces the products of the formulae VII, VIII and IX which are reproduced below and have in part good temperature stability.

The known preparation of nitrogen-containing derivatives of dibenz[c,e][1,2]-oxaphosphorin-6-oxides by aminomethylation of 6H-dibenz[c,e][1,2]-oxaphosphorin-6-oxides delivers merely a limited number of substances of this type with a restricted property profile. In addition, the danger exists with these compounds that, in the presence of water which can be easily absorbed by the phosphorus-containing ring system, they can decompose whilst releasing the 6H-dibenz[c,e][1,2]-oxaphosphorin-6-oxides. This reverse reaction can take place in particular at high temperatures, as are applied in the incorporation of flameproofing agents into thermoplastics. The thereby resulting 6H-dibenz[c,e][1,2]-oxaphosphorin-6-oxides can lead to the acidic decomposition of the thermoplastics.

The production of the nitrogen-bridged dibenz[c,e][1,2]-oxaphosphorin-6-oxides derivatives of the formulae VII, VIII and IX according to WO 2006/084488 A1 is a complicated method. One disadvantage of this method is the complex synthesis of the alkoxy-(6H)-dibenz[c,e](1,2]-oxaphosphorins required as starting substances (e.g. according to DE 102 06 982 B4). A further disadvantage resides in the fact that the alkoxy-(6H)-dibenz[c,e][1,2]-oxaphosphorins must be used in a considerable excess during the conversion with the polyols since they react only slowly with the hydroxyl groups, the almost complete conversion of which is however required. Therefore, they must be distilled off after completion of the reaction in a high vacuum, which is cost-intensive and can be achieved only with great difficulty in the production of the macromolecular dibenz[c,e][1,2]-oxaphosphorin-6-oxide derivative of formula IX. The synthesis of the polyol THIC-O which is required for the production of the derivative according to formula IX and effected by acid-catalysed oligomerisation of 1,3,5-tris(2-hydroxyethyl) isocyanuric acid (THIC) is also relatively complicated. Of particular complexity thereby is the separation, required here, of the acidic catalyst after completion of the reaction, for which the THIC-O must firstly be dissolved in a polar solvent. Altogether, the production method described in WO 2006/084488 A1 is hence not particularly suitable for application on a large scale.

It is hence the object of the present invention to develop a method which makes possible the production of sufficiently temperature-loadable derivatives of the dibenz[c,e][1,2]-oxaphosphorin-6-oxides, in particular the substances of formulae VII, VIII and IX in a simple and cost-effective way.

### BRIEF DESCRIPTION OF THE SEVERAL VIEWS OF THE DRAWINGS

FIG. 1 illustrates an embodiment of the invention, in which the nitrogen-containing alcohol IIIa and 6-(N(1-propyl)-amino)-(6H)-dibenz[c,e][1,2]-oxaphosphorin (Ia) are reacted to form the nitrogen-bridged 6H-dibenz[c,e][1,2]-phosphorin X and amine Va, which corresponds to the amine portion of Ia. R₁, R₂, and R₆ are defined herein. R₈, R₉, and R₁₀ are the same or different and mean hydrogen, linear or branched C₁-C₂₂ oxa radicals, alkylsulphonyl radicals, arylsulphonyl radicals, thioaryl radicals, thioalkyl radicals, linear or branched C₃-C₂₂ alkenyl radicals, linear or branched C₃-C₂₂ alkynyl radicals, linear or branched C₁-C₂₂ hydroxyalkyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals, C₇-C₂₂ alkylaryl radicals or a possibly substituted piperidin-4-yl group.

FIG. 2 illustrates an embodiment of the invention, in which 1,3,5-tris(2-hydroxyethyl) isocyanuric acid (THIC) (IVa) and 6-(N(1-propyl)-amino)-(6H)-dibenz [c,e][1,2]-oxaphosphorin (Ia) are reacted to form the nitrogen-bridged 6H-dibenz [c,e][1,2]-phosphorin XI and amine Va, which corresponds to the amine portion of Ia.

FIG. 3 illustrates an embodiment of the invention, in which the polyhydroxy compound THIC-O(IVb) and 6-(N(1-propyl)-amino)-(6H)-dibenz[c,e][1,2]-oxaphosphorin (Ia) are reacted to form the nitrogen-bridged 6H-dibenz[c,e][1,2]-phosphorin XII and amine Va, which corresponds to the amine portion of Ia.

### DETAILED DESCRIPTION OF THE INVENTION

This object is achieved with the features of patent claim 1, the dependent claims representing advantageous developments.

According to the invention, a method for the production of bridged dibenz[c,e][1,2]-oxaphosphorin-6-oxides is hence provided, in which a dibenz[c,e][1,2]-oxaphosphorin of the general formula I

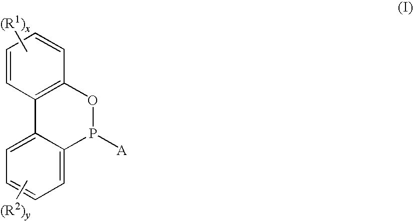

is converted with the release of a compound of the general formula HA with at least a di,-tri- and/or polyhydric alcohol, wherein independently of each other

A
is a primary amine radical, a secondary amine radical substituted in a similar or mixed manner, a heterocyclic amine radical or a hydrazine derivative,

x and y are 0, 1, 2, 3 or 4, and also

R¹ and R²
are the same or different and mean hydrogen, linear or branched C₁-C₂₂ alkyl radicals, linear or branched C₁-C₂₂ oxa radicals, alkylsulphonyl radicals, arylsulphonyl radicals, thioaryl radicals, thioalkyl radicals, linear or branched C₃-C₂₂ alkenyl radicals, linear or branched C₃-C₂₂ alkinyl radicals, linear or branched C₁-C₂₂ hydroxyalkyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals, C₇-C₂₂ alkylaryl radicals, a possibly substituted piperidin-4-yl group and/or halogen atoms.

According to the method according to the invention, nitrogen-bridged derivatives of dibenz[c,e][1,2]-oxaphosphorin-6-oxides are hence represented, such as for example the subsequently reproduced derivatives of the formulae VII-IX:

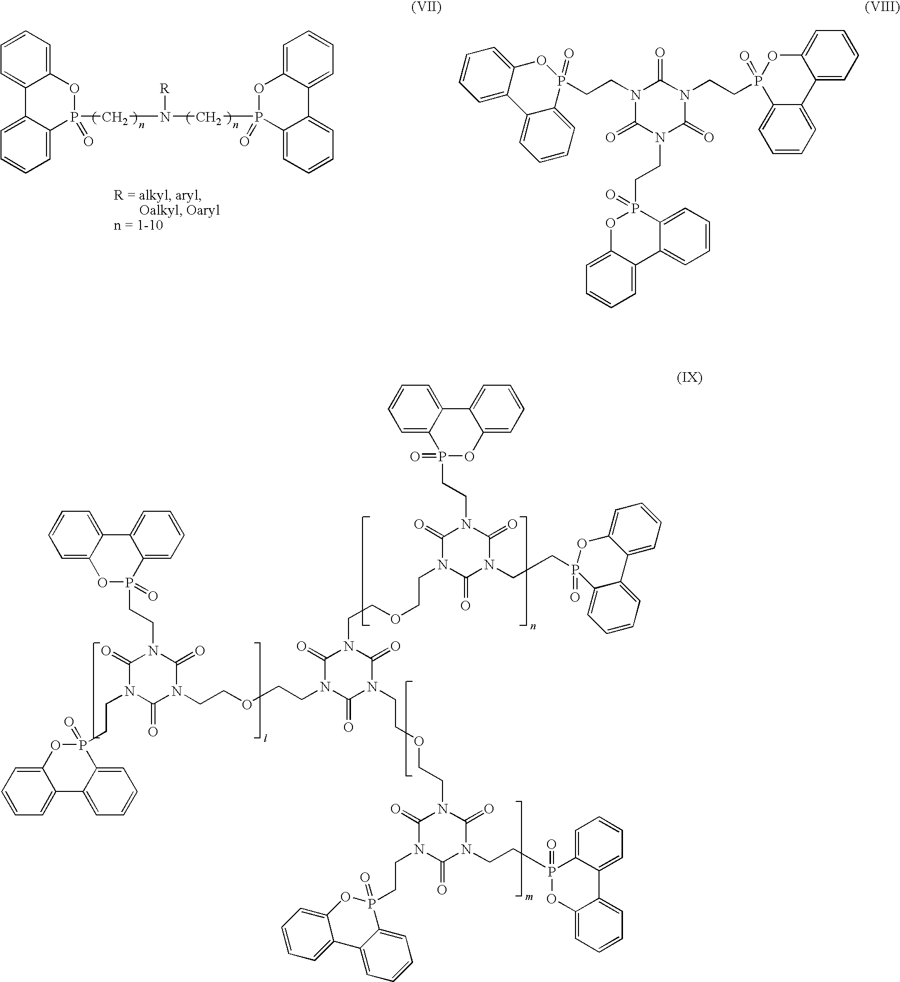

In the diagram reproduced above, R thereby means hydrogen, linear or branched C₁-C₂₂ alkyl radicals, linear or branched C₁-C₂₂ oxa radicals, alkylsulphonyl radicals, arylsulphonyl radicals, thioaryl radicals, thioalkyl radicals, linear or branched C₃-C₂₂ alkenyl radicals, linear or branched C₃-C₂₂ alkinyl radicals, linear or branched C₁-C₂₂ hydroxyalkyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals, C₇-C₂₂ alkylaryl radicals, a possibly substituted piperidin-4-yl group.

Alkylsulphonyl or arylsulphonyl radicals are also termed —SO₂-alkyl or —SO₂-aryl radicals.

There are understood by oxa radicals, radicals with an oxygen atom as bridge atom, such as e.g. —O-alkyl or —O-aryl.

It is hence essential to the invention that, as educt for the synthesis of the bridged oxaphosphorin-6-oxides, a compound according to formula I is used, the phosphorus atom being bonded directly to the nitrogen atom of a nitrogen-containing radical, i.e. for example an amine or hydrazine radical. It results as a particular advantage of the method according to the invention that the method can be implemented without solvents and without complex separation methods and purification steps, such as e.g. vacuum distillation, and hence is outstandingly suitable for application on a large scale. All the method steps taking place during the reaction can be implemented in the same reaction vessel without purification of possibly occurring intermediate products, as is explained in more detail subsequently, being required. In addition, the phosphorus-containing starting substances which are used can be produced economically from the commercially available 6-H-dibenz[c,e][1,2]-oxaphosphorin-6-oxide and are sufficiently reactive so that an excessive reagent excess is not required. A further advantage of the present invention is that the reaction is insensitive to external influences, such as e.g. traces of acid.

In an advantageous embodiment, there is used as reaction partner for the oxaphosphorin of the general formula I a dihydric alcohol of the general formula II

HO—X—OH (II)

X being selected from the group comprising linear or branched C₁-C₂₂ alkandiyl radicals, linear or branched C₃-C₂₂ alkoxycarbonylalkandiyl radicals, C₃-C₁₂ cycloalkandiyl radicals, C₆-C₁₄ arendiyl radicals, C₇-C₂₂ aralkandiyl radicals, C₇-C₂₂ alkylarendiyl radicals and nitrogen-containing radicals.

In the case of the dihydric alcohols, it is further preferred if the diol which is used concerns a nitrogen-containing diol. There is hereby used in particular an alkylaminodiol of the general formula III

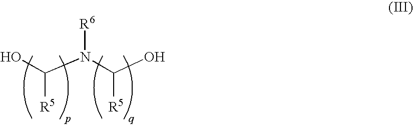

R⁵
meaning hydrogen, linear or branched C₁-C₂₂ alkyl radicals, linear or branched C₃-C₂₂ alkenyl radicals, linear or branched C₃-C₂₂ alkinyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals and/or C₇-C₂₂ alkylaryl radicals,

R⁶
meaning hydrogen, linear or branched C₁-C₂₂ alkyl radicals, linear or branched C₃-C₂₂ alkenyl radicals, linear or branched C₃-C₂₂ alkinyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals, C₇-C₂₂ alkylaryl radicals, C₂-C₂₂ aliphatic amide radicals, C₆-C₂₂ aromatic amide radicals, C₇-C₂₂ araliphatic amide radicals, C₁-C₂₂ aliphatic sulphonamide radicals, C₆-C₂₂ aromatic sulphonamide radicals or C₇-C₂₂ araliphatic sulphonamide radicals, and

p and q
being, independently of each other, from 1 to 10.

As an alternative hereto or also in addition to the dihydric alcohol, it is possible in addition to use a polyhydric alcohol of the general formula IV

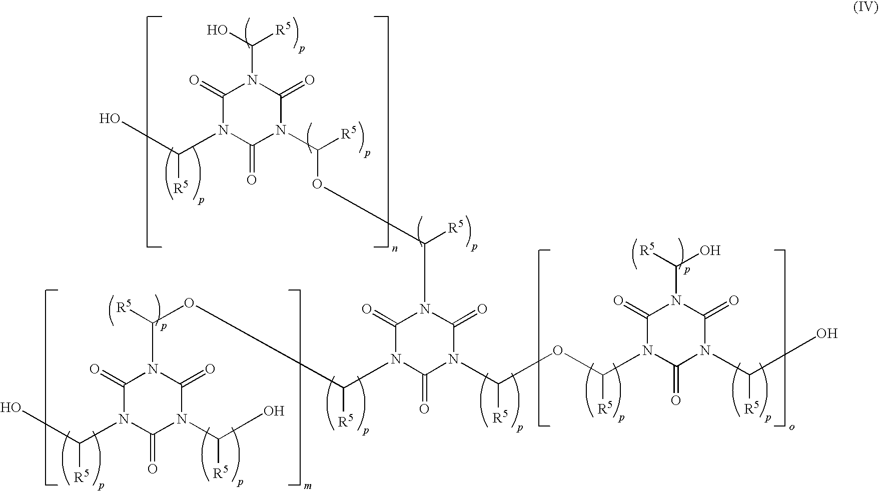

p and R⁵ hereby have the meaning indicated above. In the case of the trihydric alcohol, m, n and o are all 0. The trihydric alcohol is hence derived from cyanuric acid.

As an alternative hereto, it is however possible that the just-described alcohol is precondensed in order to obtain an oligomeric or polymeric polyol which is outstandingly suitable for cross-linking of the oxaphosphorin compounds of the general formula I. In general, a mixture of a plurality of oligomeric polyols of a different condensation degree is hereby obtained. Hence, for example the compounds of the general formula IX can then be produced. It should be preferred in particular that the mixture, containing at least one polyhydric alcohol of formula IV with (m+n+o)>1, is produced directly before the conversion with the aminated dibenz[c,e][1,2]-oxaphosphorin of the general formula I by acid-catalysed condensation reaction of the trihydric alcohol of formula IV with m=n=o=0. There is used preferably as catalyst p-toluenesulphonic acid or p-toluenesulphonic acid hydrate. The catalyst is thereby added advantageously in several portions, the already produced reaction water being respectively removed in advance from the reaction mixture. However mixtures of trihydric alcohol and the at least one polyhydric alcohol are likewise able to be used.

The method for the production of the compound of formula IX can be implemented hence for example as instillation synthesis without the resulting intermediate product, namely the above-mentioned polyhydric alcohol of formula IV, requiring to be isolated. This is extremely advantageous in particular from the point of view of the economy of the method.

A further advantage of the present invention is that the method can be implemented in the absence of a solvent.

The compound of the general formula HA, which is released by substitution during the method according to the invention and which therefore concerns an amine- or hydrazine compound, is preferably removed from the reaction mixture in order to move the reaction equilibrium advantageously towards the product side. Preferably, the removal of the compound of formula HA is effected by distilling off which takes place in particular at reduced pressure, there being understood by reduced pressure a pressure which is less than normal pressure.

The method is implemented advantageously at temperatures between 50 and 300° C., preferably between 110 and 240° C.

The reaction thereby takes place in particular in two steps,

a) in the first step, substitution of the radical A of the dibenz[c,e][1,2]-oxaphosphorin of the general formula I being effected by a di-, tri- and/or polyhydric alcohol with splitting of the amine HA and

b) in the second step at a higher temperature than in the first step, an intramolecular Michaelis-Arbuzov rearrangement taking place to form the end product.

The first step of the reaction, i.e. the substitution, is effected advantageously at 110 to 170° C., particularly preferred at 130 to 160° C.

The second step of the reaction, i.e. the intramolecular rearrangement, is preferably effected at 155 to 240° C., particularly preferred at 170 to 230° C.

Preferably, a catalyst, such as e.g. p-toluenesulphonic acid methylester, can be added to the reaction mixture of this second step, in quantities of 0.5 to 4% by mol, preferably 1 to 3% by mol, relative to the dibenz[c,e][1,2]-oxaphosphorin (I) which is used.

A condensation of the alcohol components can also precede the first step in order to obtain an oligomeric or polymeric polyol. Preferably, a catalyst in the form of an acid, such as e.g. p-toluenesulphonic acid or p-toluenesulphonic acid hydrate, can be added during this condensation. This is effected preferably in several portions. The condensation reaction is implemented at 170 to 210° C., preferably at 180 to 200° C.

The radicals A of the general formula I are thereby advantageously amine radicals of the general formula V,

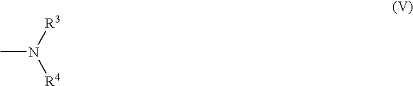

R³
meaning hydrogen, linear or branched C₁-C₂₂ alkyl radicals, linear or branched C₁-C₂₂ oxa radicals, alkylsulphonyl radicals, arylsulphonyl radicals, thioaryl radicals, thioalkyl radicals, linear or branched C₃-C₂₂ alkenyl radicals, linear or branched C₃-C₂₂ alkinyl radicals, linear or branched C₁-C₂₂ hydroxyalkyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals, C₇-C₂₂ alkylaryl radicals or a possibly substituted piperidin-4-yl group, and

R⁴
meaning linear or branched C₁-C₂₂ alkyl radicals, linear or branched C₁-C₂₂ oxa radicals, alkylsulphonyl radicals, arylsulphonyl radicals, thioaryl radicals, thioalkyl radicals, linear or branched C₃-C₂₂ alkenyl radicals, linear or branched C₃-C₂₂ alkinyl radicals, linear or branched C₁-C₂₂ hydroxyalkyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals, C₇-C₂₂ alkylaryl radicals or a possibly substituted piperidin-4-yl group.

Similarly, the radical A can also advantageously represent a hydrazine radical of the general formula VI,

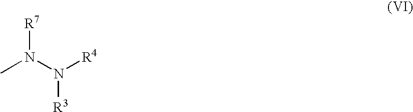

R³ and R⁴ having the above-mentioned meaning and

R⁷
meaning hydrogen, linear or branched C₁-C₂₂ alkyl radicals, linear or branched C₁-C₂₂ oxa radicals, alkylsulphonyl radicals, arylsulphonyl radicals, thioaryl radicals, thioalkyl radicals, linear or branched C₃-C₂₂ alkenyl radicals, linear or branched C₃-C₂₂ alkinyl radicals, linear or branched C₁-C₂₂ hydroxyalkyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals, C₇-C₂₂ alkylaryl radicals or a possibly substituted piperidin-4-yl group.

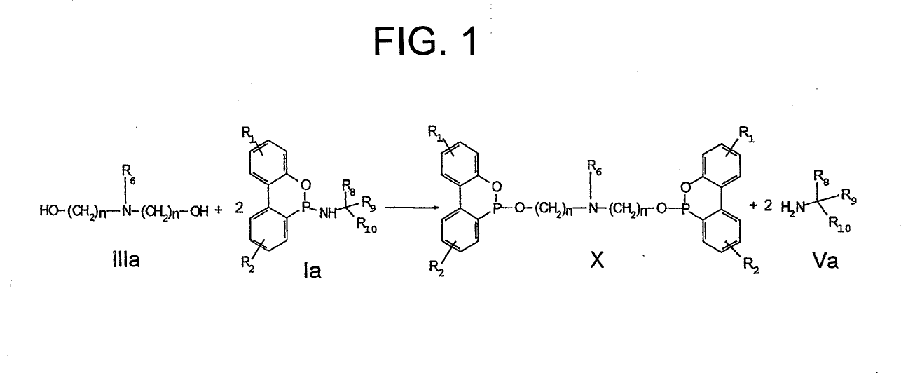

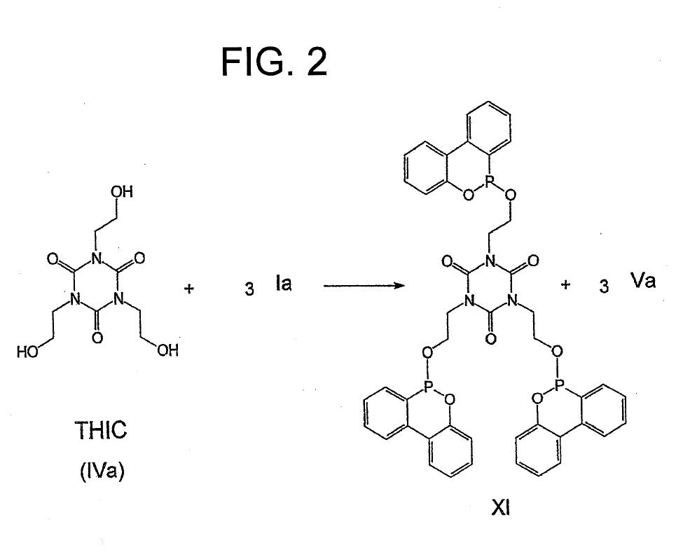

The present invention is explained in more detail with reference to FIGS. 1-3 and also examples 1 and 2 without restricting the invention to the parameters mentioned there.

In the first step of the production process which is represented with reference to three examples in FIGS. 1-3, the nitrogen-containing alcohols IIIa, IVa and IVb according to the reactions shown in FIGS. 1, 2, and 3, respectively, are converted with the 6-alkylamino-(6H)-dibenz[c,e][1,2]-oxaphosphorins Ia, the nitrogen-bridged 6H-dibenz[c,e][1,2]-phosphorins X, XI and XII being produced, which contain trivalent phosphorus and are still sensitive to hydrolysis. During the reaction, a quantity of the amine Va corresponding to the alcohol equivalent is released.

Substances X, XI, XII are converted in the second step with the help of an intramolecular Michaelis-Arbuzov reaction into the end products VII, VIII, IX. The nitrogen-bridged dibenz[c,e][1,2]-oxaphosphorin-6 oxides VII, VIII and IX are obtained with high selectivity so that purification of the products in most cases is unnecessary.

The 6-alkyl-amino-(6H)-dibenz[c,e][1,2]-oxaphosphorins Ia required for the reactions (FIGS. 1-3) are produced corresponding to the methods known from the literature or are produced by the aminolysis of 6-chloro-(6H)-dibenz[c,e][1,2]-oxaphosphorins which has been known for a fairly long time (EP 0 005 441 A1, JP 54138565 AA).

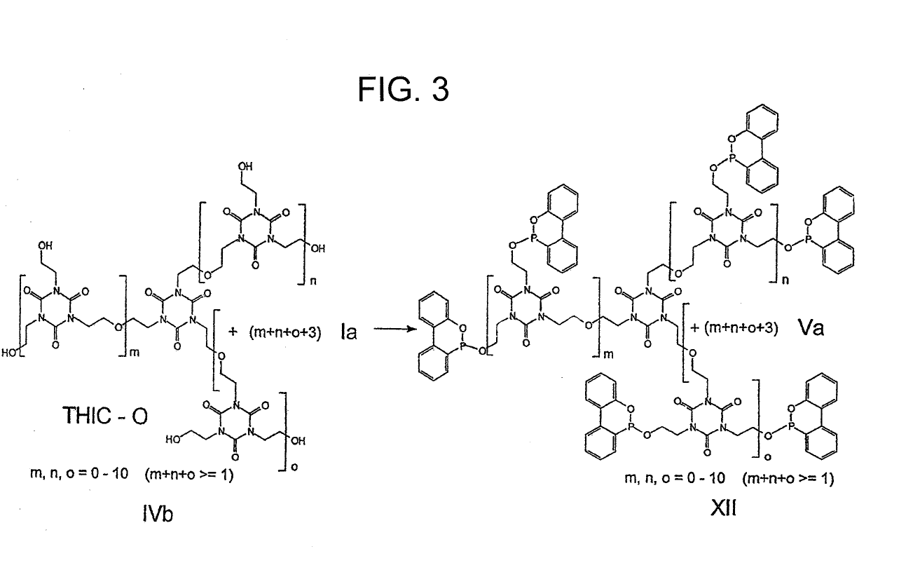

The polyhydroxy compound THIC-O(IVb) required as starting substance for the reaction (FIG. 3) is produced corresponding to WO 2006/084488 by oligeromerisation of 1,3,5-tris(2-hydroxyethyl) isocyanuric acid (THIC) (IVa). In contrast to the mentioned publication, the complex separation of the acidic catalyst is however dispensed with so that the THIC-O(IVb) can be converted immediately after production thereof with the 6-alkylamino-(6H)-dibenz[c,e][1,2]-oxaphosphorins Ia corresponding to the reaction shown in FIG. 3.

Starting from THIC(IVa), the entire production of the substance IX is hence implemented as an uninterrupted and solvent-free process in the same reaction vessel, processing of the intermediate steps or the purification of intermediate products (e.g. XII) not being required.

The alkylamino-(6H)-dibenz[c,e][1,2]-oxaphosphorins Ia used here for conversion of the polyols can be produced effectively from commercially available 6H-dibenz[c,e][1,2]-oxaphosphorin-6-oxides, no unusable by-products being produced. They have a much higher reactivity than the alkoxy-(6H)-dibenz[c,e][1,2]-oxaphosphorins used to date (WO 2006/084488 A1) so that a very high conversion of the hydroxyl groups is achieved even with stoichiometric reagent use and no excess phosphorus compound requires to be distilled off, as is required in the known method (WO 2006/084488 A1).

Hence the substances VII, VIII and IX can be produced substantially more easily, more economically and essentially on a larger scale than previously. The production of the macromolecular substance IX is simplified in particular. There is the advantage here in addition that the acidic catalyst which is used in the synthesis of THIC-O(IVb) need not be separated because it does not disrupt the conversion with the phosphorus compound. A catalyst separation is however absolutely necessary in the previous method according to WO 2006/084488 A1, for which purpose the THIC-O is firstly dissolved in a polar solvent which must be removed again in its entirety subsequent to this method step. Because of the mentioned improvements, the entire production process of IX starting from the THIC can now be implemented in one reaction vessel, without solvents, without purification of the intermediate products and on a large scale.

### EXAMPLES

### Example 1

### Production of Compound VIII

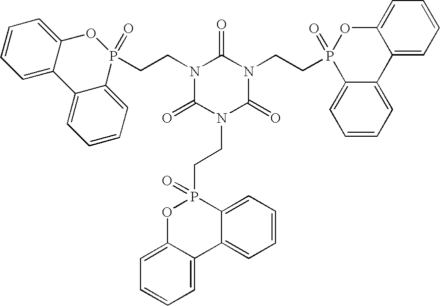

In a vacuum-tight glass apparatus which is equipped with a sturdy agitator, a thermometer, an inert gas supply pipe and also with a heating bath, 32.67 g water-free 1,3,5-tris(2-hydroxyethyl)-isocyanuric acid (THIC) are heated. After melting of the THIC, agitation is begun and the temperature of the heating bath is lowered to 135° C. Thereafter, 96.5 g 6-(N(1-propyl)-amino)-(6H)-dibenz[c,e][1,2]-oxaphosphorin Ia heated in advance to approx. 120° C. are added. A few minutes after addition of this reagent, the pressure in the reaction vessel is carefully lowered, the reaction mixture beginning to foam. When the foaming stops, the pressure is further reduced slowly until finally 2 to 5 mbar are achieved. Now the agitation is continued at this vacuum and at a temperature of 130 to 135° C., a homogeneous melt being produced gradually from the two-component mixture. The 1-propylamine resulting during this conversion is condensed in a vacuum trap. The progress of the reaction can be followed well by means of the ¹H— and ³¹P-NMR spectra. At a conversion of 93 to 95% by mol which is achieved after approx. 15 h, the reaction temperature is increased to 140 to 142° C. It is left unchanged at this level until approx. 97% by mol of the free OH groups are converted and finally is increased to 150° C. The reaction is continued until at most 1.5% by mol free OH groups are still present in the melt. When the required conversion has been achieved, which is the case after in total approx. 24 h, the vacuum is removed by supplying argon or nitrogen. Then the melt is heated to 175° C. and 0.0075 mol (1.34 g) p-toluenesulphonic acid methylester are added. The melt is thereafter agitated further under normal pressure at a temperature of 175 to 178° C. The progress of the reaction is followed further by means of NMR spectroscopy. With increasing conversion, the viscosity of the melt greatly increases so that it is eventually barely still able to be agitated. When the rearrangement is complete which is achieved after approx. 18 to 20 h, the melt is heated up to 220° C. until discharge and is kept for another 2 h at this temperature and then poured into a steel tank in which it solidifies to form a brittle glass-like solid material which produces a white powder during grinding. The purity of the thus-produced product is approx. 95% by mol (as a mixture of three stereoisomers). The product contains less than 0.7% by mol non-converted phosphorus compound Ia.

### Example 2

### Production of Substance IX

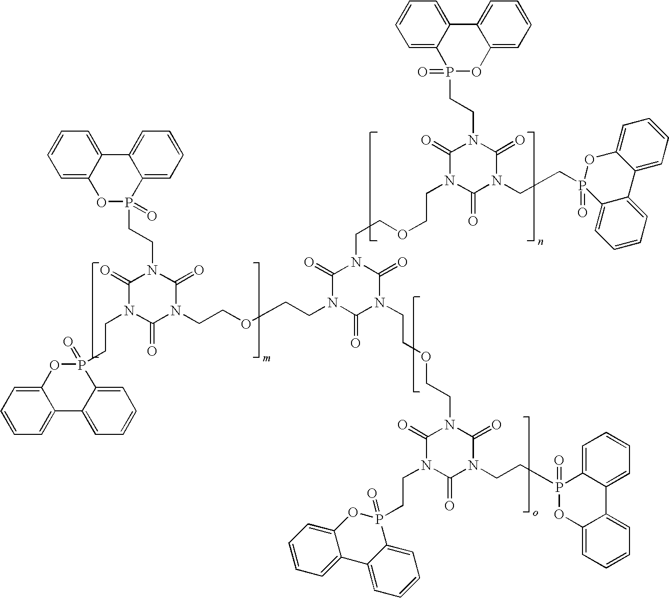

### Apparatus:

4 1 four-neck round flask which is equipped with the following components:
sturdy glass agitator, installed to be vacuum-tight,
internal thermometer,
inert gas connection,
Liebig condenser with coolable receiving vessel
heating bath
vacuum pump with vacuum trap

In a four-neck flask filled with argon or nitrogen, a mixture comprising 1131 g (4.33 mol) 1,3,5-tris(2-hydroxyethyl) isocyanuric acid (THIC, IVa) and also 1.52 g p-toluenesulphonic acid hydrate is added, melted and heated with agitation to 185° C. After a reaction duration of 3 h, the pressure is lowered to approx. 50 mbar (in approx. 5 min) in order to distil off the resulting water. Subsequently, inert gas is supplied again and the second portion of the sulphonic acid (0.6 g) is added. Then the melt is agitated for a further 8 h at 185° C. Thereafter, the resulting water is removed at approx. 20 mbar and, after filling the apparatus with inert gas, another 0.3 g of the catalyst is added. Subsequent thereto, the temperature of the melt is increased up to 193° C. and agitated for a further 3 h. At this temperature (193° C.), another two further catalyst additions are effected later at intervals of respectively 3 h (0.3 g and 0.15 g). A vacuum is applied respectively for a short time in advance. At equal intervals, samples are also removed and examined by means of NMR spectroscopy (solvent DMSO-d₆). The progress of the oligomerisation can be detected best in the changes in the ¹³C-spectra. In the case of monomeric THIC, the peaks of the aliphatic C-atoms are at 44.16 and 57.41 ppm and that of the aromatic C-atoms at 149.4 ppm. By means of the oligomerisation, two new peaks are produced for the aliphatic C-atoms at 41.5 ppm or 66.7 ppm and, for the aromatic C-atoms, three further peaks at 149.1, 149.2 and 149.3 ppm. If the oligomers comprise on average four to five THIC units, which is the case after a reaction duration of 15 to 20 h, the apparatus is evacuated up to a pressure of approx. 1 mbar. Agitation takes place for another 15 min at this vacuum and unchanged temperature, any traces of water still contained being distilled off. The melt is then cooled in the course of 1 h to 165° C. and the apparatus is subsequently filled again with inert gas. Thereafter the agitator is switched off and 1657 g distilled 6-(N(1-propyl)amino)-(6H)-dibenz[c,e][1,2]-oxaphosphorin Ia, preheated to approx. 110° C., are added. After addition of the reagent, the temperature of the mixture should be approx. 124 to 127° C. (correspondingly adapted to the oil bath temperature). If this parameter is reached, the pressure is lowered again to approx. 1 mbar and the agitator is started carefully. The mixture is initially non-homogeneous, the phase of the oligomeric THIC being very viscous. The reaction is effected initially only slowly but soon accelerates because the mutual solubility of the phases increases, which can be detected in greater foaming. If this is the case, the temperature is lowered slightly to 117 to 120° C. and the speed of rotation of the agitator is carefully increased. After approx. 1.5 h, the mixture is emulsion-like and, after approx. 2 h, completely homogeneous and relatively fluid. The 1-propylamine which is released during the conversion is condensed in a high vacuum trap. This is emptied at intervals of respectively 2 to 3 h, the normal pressure being reestablished firstly by introducing inert gas. Samples are also respectively removed for the NMR spectroscopy (the ¹H- and ³¹P-spectrum are required for detection of the conversion). The subsequent evacuation must be effected carefully since strong foaming occurs. If material reaches the upper regions of the flask due to the foaming and solidifies there, this must be melted carefully with a blast of hot air. After approx. 7 h, the conversion of Ia is approx. 70% by mol and the melt is significantly more viscous. In the course of the next approx. 7 h, the temperature is now increased continuously up to 137° C. so that a conversion of OH groups of 98% by mol is achieved. If the excess of OH groups exceeds 2% by mol, some Ia is added in addition. Thereafter, the temperature is increased continuously to 155° C. within 5 h in order to complete the conversion. If the conversion of OH groups is at least 98.5% by mol, the vacuum is removed in that nitrogen or argon is introduced into the apparatus. The melt is then heated up to approx. 175° C. and 21 g (0.1127 mol) of the rearrangement catalyst p-toluenesulphonic acid methylester are added. The initially greatly viscous melt is agitated at a temperature of 175 to 178° C. until the rearrangement degree is approx. 80% (after approx. 12 h; detectable by evaluation of the ³¹P-spectra; during the rearrangement, firstly significant quantities of 6H-dibenz[c,e][1,2]-oxaphosphorin-6-oxide are produced. However the concentration thereof reduces significantly again at the end of the reaction). The melt then becomes significantly more viscous and hence the reaction temperature begins to increase gradually and uniformly. This temperature increase is necessary since the melt viscosity increases further with progressing reaction. If 95% by mol of the phosphorus compound is rearranged, the reaction temperature should be 186 to 188° C. (after in total approx. 15 h). This temperature is maintained for another 2 h and then it is increased within 3 h up to 225° C. The again more fluid melt is then poured into a steel tank. The solidified product IX is glass-like. After rough comminution, it is ground into a white, odour-free powder. It contains approx. 93% by mol of the target product IX and on average four to five THIC units per molecule (M_{w} approx. 2200 g/mol).

1. Method for the production of bridged dibenz[c,e][1,2]-oxaphosphorin-6-oxides, a dibenz[c,e][1,2]-oxaphosphorin of the general formula I 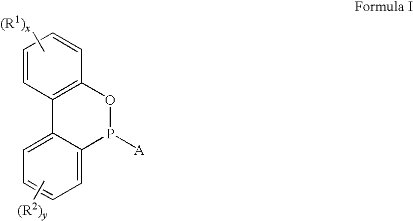
being converted with the release of a compound of the general formula HA with at least a di-, tri- and/or polyhydric alcohol, wherein independently of each other
A
is a primary amine radical, a secondary amine radical substituted in a similar or mixed manner, a heterocyclic amine radical or a hydrazine derivative,
x and y are 0, 1, 2, 3 or 4, and also
R¹ and R²
are the same or different and mean hydrogen, linear or branched C₁-C₂₂ alkyl radicals, linear or branched C₁-C₂₂ oxa radicals, alkylsulphonyl radicals, arylsulphonyl radicals, thioaryl radicals, thioalkyl radicals, linear or branched C₃-C₂₂ alkenyl radicals, linear or branched C₃-C₂₂ alkinyl radicals, linear or branched C₁-C₂₂ hydroxyalkyl radicals, linear or branched C₃-C₂₂ alkoxycarbonylalkyl radicals, C₃-C₁₂ cycloalkyl radicals, C₅-C₁₄ aryl radicals, C₇-C₂₂ aralkyl radicals, C₇-C₂₂ alkylaryl radicals, a possibly substituted piperidin-4-yl group and/or halogen atoms.

2. Method according to claim 1, characterised in that the alcohol is a dihydric alcohol of the general formula II
HO—X—OH Formula II
X being selected from the group comprising linear or branched C₁-C₂₂ alkandiyl radicals, linear or branched C₃-C₂₂ alkoxycarbonylalkandiyl radicals, C₃-C₁₂ cycloalkandiyl radicals, C₆-C₁₄ arendiyl radicals, C₇-C₂₂ aralkandiyl radicals, C₇-C₂₂ alkylarendiyl radicals and nitrogen-containing radicals.

3. Method according to the preceding claim, characterised in that the dihydric alcohol is an alkylaminodiol of the general formula III 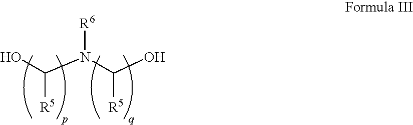
R⁵
meaning hydrogen, linear or branched C₁-C₂₂ alkyl radicals, linear or branched C₃-C₂₂ alkenyl radicals, linear or branched C₃-C₂₂ alkinyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals and/or C₇-C₂₂ alkylaryl radicals,
R⁶
meaning hydrogen, linear or branched C₁-C₂₂ alkyl radicals, linear or branched C₃-C₂₂ alkenyl radicals, linear or branched C₃-C₂₂ alkinyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals, C₇-C₂₂ alkylaryl radicals, C₂-C₂₂ aliphatic amide radicals, C₆-C₂₂ aromatic amide radicals, C₇-C₂₂ araliphatic amide radicals, C₁-C₂₂ aliphatic sulphonamide radicals, C₆-C₂₂ aromatic sulphonamide radicals or C₇-C₂₂ araliphatic sulphonamide radicals, and
p and q
being, independently of each other, from 1 to 10.

4. Method according to one of the preceding claims, characterised in that the alcohol is a trihydric alcohol of formula IV with m=n=o=0, R⁵ and p having the above-indicated meaning and/or being a mixture containing at least one polyhydric alcohol of the general formula IV with (m+n+o)>1, preferably 30≧(m+n+o)>1 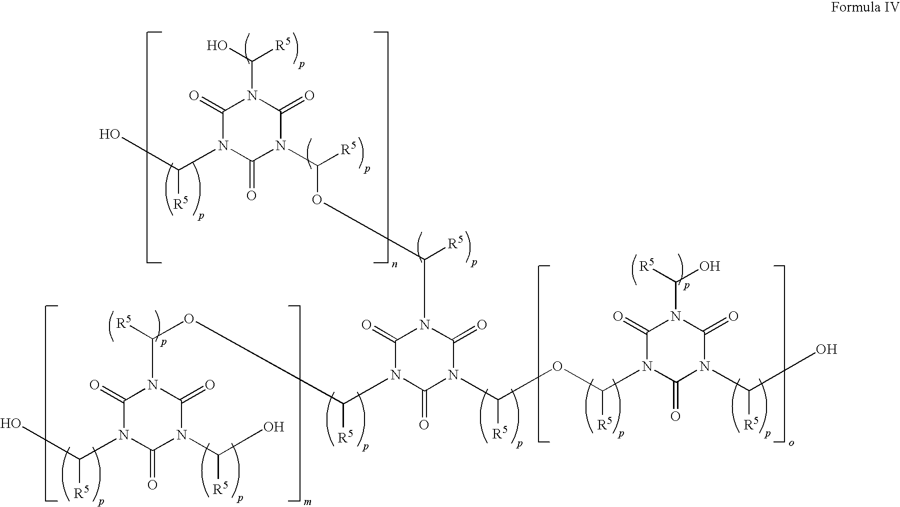
p and R⁵ having the above-indicated meaning
and
m, n and o
being, independently of each other, 0 to 10.

5. Method according to the preceding claim, characterised in that the mixture, containing at least one polyhydric alcohol of formula IV with (m+n+o)>1, is produced directly before the conversion with the aminated dibenz[c,e][1,2]-oxaphosphorin of the general formula I by acid-catalysed condensation reaction of the trihydric alcohol of formula IV with m=n=o=0, preferably under the catalytic effect of at least one acid, in particular p-toluenesulphonic acid.

6. Method according to the preceding claim, characterised in that the production of the mixture, containing at least one polyhydric alcohol of formula IV (m+n+o)>1 and the conversion of the mixture, containing at least one polyhydric alcohol of formula IV (m+n+o)>1, is implemented as instillation synthesis with the aminated dibenz[c,e][1,2]-oxaphosphorin of the general formula I.

7. Method according to one of the preceding claims, characterised in that it is implemented in the absence of a solvent.

8. Method according to one of the preceding claims, characterised in that the compound of the general formula HA produced during conversion is distilled off, preferably at reduced pressure.

9. Method according to one of the preceding claims, characterised in that the method is implemented at temperatures between 50 and 300° C., preferably between 110 and 240° C.

10. Method according to one of the preceding claims, characterised in that the conversion is effected in two steps,
a) in the first step, substitution of the radical A of the dibenz[c,e][1,2]-oxaphosphorin of the general formula I being effected by a di-, tri- and/or polyhydric alcohol with splitting of the amine HA and
b) in the second step at a higher temperature than in the first step, an intramolecular Michaelis-Arbuzov rearrangement taking place to form the end product.

11. Method according to the preceding claim, characterised in that, in the first step, a catalyst selected from the group comprising p-toluenesulphonic acid and p-toluenesulphonic acid hydrate is added.

12. Method according to one of the two preceding claims, characterised in that, in the second step, p-toluenesulphonic acid methylester is used as catalyst.

13. Method according to one of the preceding claims, characterised in that the radical A represents an amine radical of the general formula V 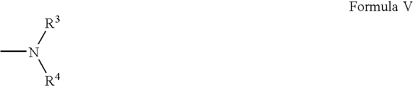
R³
meaning hydrogen, linear or branched C₁-C₂₂ alkyl radicals, linear or branched C₁-C₂₂ oxa radicals, alkylsulphonyl radicals, arylsulphonyl radicals, thioaryl radicals, thioalkyl radicals, linear or branched C₃-C₂₂ alkenyl radicals, linear or branched C₃-C₂₂ alkinyl radicals, linear or branched C₁-C₂₂ hydroxyalkyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals, C₇-C₂₂ alkylaryl radicals or a possibly substituted piperidin-4-yl group, and
R⁴
meaning linear or branched C₁-C₂₂ alkyl radicals, linear or branched C₁-C₂₂ oxa radicals, alkylsulphonyl radicals, arylsulphonyl radicals, thioaryl radicals, thioalkyl radicals, linear or branched C₃-C₂₂ alkenyl radicals, linear or branched C₃-C₂₂ alkinyl radicals, linear or branched C₁-C₂₂ hydroxyalkyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals, C₇-C₂₂ alkylaryl radicals or a possibly substituted piperidin-4-yl group.

14. Method according to one of the preceding claims, characterised in that the radical A represents a hydrazine radical of the general formula VI 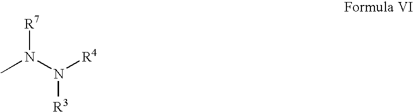
R³ and R⁴ having the above-mentioned meaning and
R⁷
meaning hydrogen, linear or branched C₁-C₂₂ alkyl radicals, linear or branched C₁-C₂₂ oxa radicals, alkylsulphonyl radicals, arylsulphonyl radicals, thioaryl radicals, thioalkyl radicals, linear or branched C₃-C₂₂ alkenyl radicals, linear or branched C₃-C₂₂ alkinyl radicals, linear or branched C₁-C₂₂ hydroxyalkyl radicals, linear or branched C₃-C₂₂ alkoxycarbonylalkyl radicals, C₃-C₁₂ cycloalkyl radicals, C₆-C₁₄ aryl radicals, C₇-C₂₂ aralkyl radicals, C₇-C₂₂ alkylaryl radicals or a possibly substituted piperidin-4-yl group.
